(19) United States
(12) Patent Application Publication
(10) Pub. No.: US 2023/0310406 A1
(43) Pub. Date: Oct. 5, 2023

(54) TREATMENT OF NEUROLOGICAL DISORDERS

(71) Applicant: BioCorRx, Inc., Anaheim, CA (US)

(72) Inventors: Brady J. Granier, Anaheim, CA (US); Jeffrey M. Witkin, Carmel, IN (US); David Martirosyan, Lake Forest, CA (US)

(21) Appl. No.: 18/327,552

(22) Filed: Jun. 1, 2023

Related U.S. Application Data
(63) Continuation of application No. PCT/US2021/061285, filed on Nov. 30, 2021.
(60) Provisional application No. 63/119,843, filed on Dec. 1, 2020.

(51) Int. Cl. A61K 31/4468

(52) U.S. Cl. CPC A61K 31/4468

(57) ABSTRACT

The present disclosure is directed toward uses of compound 1 (or its stereoisomers) and compositions and/or dosage forms containing compound 1, wherein compound 1 is N-[2,5-dimethyl-1-(2-phenylethyl)piperidin-4-yl]-N-phenylpropanamide.

### CROSS-REFERENCE TO RELATED APPLICATIONS

This application is a continuation application of International Patent Application No. PCT/US2021/061285, filed Nov. 30, 2021, which claims the benefit of U.S. Provisional Patent Application 63/119,843, filed Dec. 1, 2020, which is incorporated by reference herein in its entirety.

### BACKGROUND

Opium is one of the world's oldest drugs. Opium derivatives morphine and codeine are among the most used clinical drugs to relieve severe pain, but addiction to and abuse of these medicines have inspired research into new pain relievers lacking side effects. The search for novel modulators of opioid receptors, particularly those that may be non-addictive or that do not bring about withdrawal symptoms, are an active area of unmet medical need.

### SUMMARY

The present disclosure includes pharmaceutical compositions comprising compound 1 for the treatment and/or prevention of diseases and disorders. Compound 1 modulates various opioid receptors and may be useful in the treatment of various medical disorders and diseases.

Some embodiments include a method of treating a disease or disorder, comprising administering compound 1 to a human being in need thereof, wherein compound 1 is represented by the formula:

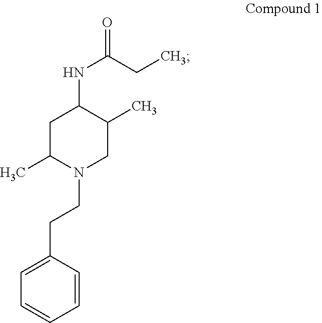 [SMILES: CCC(=O)NC1CC(C)N(CCc2ccccc2)CC1C]

or a pharmaceutically acceptable salt thereof, wherein the disease or disorder is not opioid intoxication, opioid use disorder, opioid overdose, narcotic depression, septic shock, alcoholism, schizophrenia, pain, depression, obesity or weight-gain.

### DETAILED DESCRIPTION

Compound 1 has the general formula shown below.

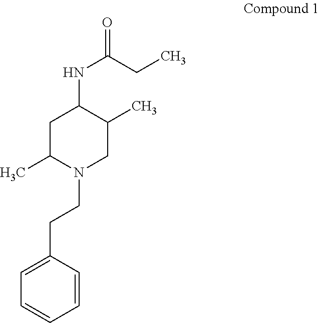

Compound 1 is known as 1-(2-phenylethyl)2,5-dimethyl-4-propionilaminopiperidine, or N-[2,5-dimethyl-1-(2-phenylethyl)piperidin-4-yl]propanamide, or 2,5-dimethyl-1-(2-phenyl)ethyl-4-propionilaminopiperidine or N-(2,5-dimethyl-1-phenethylpiperidin-4-yl)propionamide.

Unless otherwise indicated, any reference to a compound 1 herein, by structure, name, or any other means, includes pharmaceutically acceptable salts; alternate solid forms, such as polymorphs, solvates, hydrates, etc.; stereoisomers; tautomers; deuterium-modified compounds, such as deuterium modified compound 1; or any chemical species that may rapidly convert to a compound described herein under conditions in which the compounds are used as described herein.

A “pharmaceutically acceptable salt” retains the desirable biological activity of compound 1 without unacceptable toxicological effects. Salts can be salts with a suitable acid, including, but not limited to, hydrochloric acid, hydrobromic acid, sulfuric acid, phosphoric acid, nitric acid and the like; acetic acid, oxalic acid, tartaric acid, succinic acid, malic acid, benzoic acid, pamoic acid, alginic acid, methanesulfonic acid, naphthalenesulphonic acid, and the like. Also included are salts of other acids such as salts with trifluoroacetic acid, chloroacetic acid, and trichloroacetic acid.

“Opiate” is a specific term that is used to describe drugs (natural and semi-synthetic) derived from the juice of the opium poppy. For example, morphine is an opiate but fentanyl (a completely synthetic drug) is not. “Opioid” is a general term that includes naturally occurring, semi-synthetic, and synthetic drugs, which produce their effects by combining with opioid receptors and are competitively antagonized by naloxone. For the purposes of this disclosure, the term opioid refers to opioid agonists, opioid antagonists, opioid peptides, and opioid receptors.

Superficially, compound 1 resembles the structure of fentanyl, which is a well-known pain medication and a very potent opioid receptor agonist. Compound 1, in contrast, was found to be a potent antagonist of the effects of fentanyl (respiratory depression and analgesia) that are induced by fentanyl through the mu opiate receptor. Further in vitro analyses have shown that compound 1 has a unique pharmacological profile. Specifically, compound 1 is found to be a partial agonist of mu opiate (MOP) receptors, an antagonist at kappa (KOP) and ORL1 (NOP) receptors, with further activity at sigma opiate sites. A potentially important property of partial agonists is that they display both agonistic and antagonistic effects. In the presence of a full agonist, a partial agonist will act as an antagonist, competing with the full agonist for the same receptor and thereby reducing the ability of the full agonist to produce its maximum effect. It is believed that the antagonistic nature of compound 1 at the ORL1 receptor may distinguish its activity in the context of existing medicines. A summary of the in vitro pharmacology of compound 1 in relation to common standard-of-care medications for opioid intoxication is shown below in Table 1.

TABLE 1

| | Receptor | | | | |
|---|---|---|---|---|---|
| | Mu | Kappa | Delta | ORL1 | |
| Cmpd | (MOP) | (KOP) | (DOP) | (NOP) | Sigma |
| Compound 1 | Partial | Antagonist | Antagonist | Antagonist | High |
| | agonist | | but weak | | Affinity |
| Buprenorphine | Partial | Antagonist | Antagonist | Agonist | High |
| | agonist | | but weak | | Affinity |
| Naloxone | Antagonist | Antagonist | Antagonist | Weak | Weak |
| Naltrexone | Antagonist | Antagonist | Antagonist | Weak | Weak |
| | | but weak | but weak | | |

Compound 1 also displays in vitro antagonistic activity at adrenergic receptors Alpha 1B, 2B and 2C, histamine H1 receptor, cholinergic muscarinic receptors 1, 3, 4, and 5, dopamine D4 receptor, and serotonin receptors 1A and 1B.

Compound 1 may be used alone or in combination with other drugs for the treatment of various conditions. In some embodiments, compound 1 is used alone and/or compound 1 is the only drug or active pharmaceutical ingredient in the pharmaceutical composition, medicament, or dosage form. In some embodiments, compound 1 is used in combination with another drug. In some embodiments, compound 1 and another drug or active pharmaceutical ingredient are both present in the pharmaceutical composition, medicament, or dosage form.

Pharmaceutical compositions, medicaments, or dosage forms comprising compound 1 may contain about 0.01 mg to about 1000 mg, 0.01-0.1 mg, 0.1-0.2 mg, 0.2-0.3 mg, 0.3-0.4 mg, 0.4-0.5 mg, 0.5-0.6 mg, 0.6-0.7 mg, 0.7-0.8 mg, 0.8-0.9 mg, 0.9-1 mg, about 1-2 mg, about 2-3 mg, about 3-4 mg, about 4-5 mg, about 5-6 mg, about 6-7 mg, about 7-8 mg, about 8-9 mg, about 9-10 mg, about 10-11 mg, about 11-12 mg, about 12-13 mg, about 13-14 mg, about 14-15 mg, about 15-16 mg, about 16-17 mg, about 17-18 mg, about 18-19 mg, about 19-20 mg, about 20-22 mg, about 22-24 mg, about 24-26 mg, about 26-28 mg, about 28-30 mg, about 30-32 mg, about 32-34 mg, about 34-36 mg, about 36-38 mg, about 38-40 mg, about 40-42 mg, about 42-44 mg, about 44-46 mg, about 46-48 mg, about 48-50 mg, about 50-55 mg, about 55-60 mg, about 60-65 mg, about 65-70 mg, about 70-75 mg, about 75-80 mg, about 80-85 mg, about 85-90 mg, about 90-95 mg, about 95-100 mg, about 100-110 mg, about 110-120 mg, about 120-130 mg, about 130-140 mg, about 140-150 mg, about 150-160 mg, about 160-170 mg, about 170-180 mg, about 180-190 mg, about 190-200 mg, about 200-300 mg, about 300-400 mg, about 400-500 mg, about 500-600 mg, about 600-700 mg, about 700-800 mg, about 800-900 mg, about 900-1000 mg, about 10-200 mg, about 10-100 mg, 200-500 mg, about 500-1000 mg, or about 200 mg, about 300 mg, about 400 mg, about 1000 mg, or any amount in a range bounded by any of these values, of compound 1 in a free base form or a salt form.

Data obtained from cell culture assays or animal studies can be used in formulating a range of dosage for use in humans. If the medicament is provided systemically, the dosage of such compounds lies preferably within a range of circulating concentrations that include the IC₅₀ or EC₅₀ with little or no toxicity. The dosage may vary within this range depending upon the dosage form employed and the route of administration utilized. For any compound used in the method described herein, the therapeutically effective dose can be estimated initially from cell culture assays. A dose may be formulated in animal models to achieve a circulating plasma concentration or within the local environment to be treated in a range that includes the IC50 (i.e., the concentration of the test compound that achieves a half-maximal inhibition of an opiate receptor) as determined in cell culture. Such information can be used to more accurately determine useful doses in humans. Levels in plasma may be measured, for example, by liquid chromatography coupled to mass spectrometry. In some embodiments, a modulating effect is achieved with an IC50 less than 100 μM, 50 μM, 30 μM, 20 μM, 10 μM, 5 μM, or 1 μM.

An “effective amount” is an amount sufficient to effect beneficial or desired results. For example, a therapeutic amount is one that achieves the desired therapeutic effect. This amount can be the same or different from a prophylactically effective amount, which is an amount that prevents onset of disease or disease symptoms.

For the purposes of this disclosure, the term “treat,” “treating,” or a similar term, includes cure, mitigation, treatment, or prevention of disease in man or other animals, or any other effect that would be associated with a “drug” as defined under 21 USC 321(g).

In some embodiments, administration of compound 1 may occur one or more times per day. Some embodiments include administration of compound 1 once per day, twice per day, three times per day, or four times per day. Administration of compound 1 may occur one or more times for a single day, or for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 100, or more consecutive days. In some embodiments, administration of compound 1 may occur for up to about 7 days, up to about 14 days, up to about 1 month, up to about 6 months, up to about 1 year, up to about 2 years, up to about 5 years, up to about 10 years, up to about 20 years, up to about 50 years, or up to about 100 years. In some embodiments, administration of compound 1 is at least daily for at least two consecutive days. In some examples, administration of compound 1 is at least daily for at least seven consecutive days. Some embodiments include administration of compound 1 is at least daily for at least 14 consecutive days. In some cases, compound 1 is administered at least for 30 consecutive days.

Compound 1 may be combined with a pharmaceutical carrier selected on the basis of the chosen route of administration and standard pharmaceutical practice as described, for example, in Remington's Pharmaceutical Sciences, 2005. The relative proportions of active ingredient and carrier may be determined, for example, by the solubility and chemical nature of the compounds, chosen route of administration and standard pharmaceutical practice.

Therapeutic compounds may be administered by any means that may result in the contact of the active agent (compound 1) with the desired site or site(s) of action in the body of a patient. The compounds may be administered by any conventional means available for use in conjunction with pharmaceuticals, either as individual therapeutic agents or in a combination of therapeutic agents. For example, compound 1 may be administered as the sole active agent in a pharmaceutical composition, or compound 1 can be used in combination with other therapeutically active ingredients. In some embodiments, compound 1 is administered without an opioid and/or the dosage form containing compound 1 does not also contain an opioid.

Compound 1 may be administered to a human patient in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, sublingual and buccal; topically including dermal, rectal, and nasal inhalation via insufflation or aerosol.

Compound 1 may be formulated for oral administration, for example, with an inert diluent or with an edible carrier, or it may be enclosed in hard or soft shell gelatin capsules, compressed into tablets, or incorporated directly with the food of the diet. For oral therapeutic administration, a pharmaceutical composition comprising compound 1 may be incorporated with an excipient (such as dicalcium phosphate) and used in the form of ingestible tablets, buccal tablets, troches, capsules, elixirs, suspensions, syrups, wafers, and the like. Tablets, troches, pills, capsules and the like comprising compound 1 may also contain one or more of the following: a binder; an excipient; a disintegrating agent; a diluent; a lubricant; and a sweetening and/or flavoring agent.

When the dosage unit form of compound 1 is a capsule, it may contain, in addition to materials of the above type, a liquid carrier. Various other materials may be present as a coating, for example, tablets, pills, or capsules may be coated with shellac, sugar or both. A syrup or elixir may contain compound 1, a sweetening agent, methyl and propylparabens as preservatives, a dye and a flavoring agent. It may be desirable for material in a dosage form or pharmaceutical composition to be pharmaceutically pure and substantially nontoxic in the amounts employed.

Many embodiments of the pharmaceutical compositions comprising compound 1 utilize a binder. In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, sugar, gum tragacanth, and acacia.

Various embodiments of the pharmaceutical compositions of compound 1 also include a lubricant. Suitable lubricants include magnesium stearate, stearic acid, sodium stearyl fumarate, calcium stearate, hydrogenated vegetable oil, mineral oil, polyethylene glycol, polyethylene glycol 4000-6000, talc, glyceryl behenate, etc.

In some examples, disintegrating agents in the pharmaceutical compositions include, but are not limited to, agar, calcium carbonate, maize (corn) starch, potato starch, tapioca starch, alginic acid, alginates, certain silicates, and sodium carbonate. Suitable super disintegrating agents include, but are not limited to crospovidone, croscarmellose sodium, AMBERLITE (Rohm and Haas, Philadelphia, Pa.), and sodium starch glycolate.

In certain embodiments, diluents in the pharmaceutical compositions include, but are not limited to, mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

Some embodiments of the pharmaceutical compositions include sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the pharmaceutical compositions described herein include, but are not limited to, maltol, vanillin, ethyl vanillin, menthol, citric acid, fumaric acid, ethyl maltol, tartaric acid, peppermint, oil of wintergreen, cherry flavoring or orange flavoring.

Some pharmaceutical compositions or dosage forms may be a liquid or may comprise a solid phase dispersed in a liquid. In some embodiments, compound 1 may be formulated for parental or intraperitoneal administration. Solutions of compound 1 as its free base or pharmaceutically acceptable salts can be prepared in water suitably mixed with a surfactant, such as hydroxypropylcellulose. In certain embodiments, a pharmaceutical composition may be a dispersion that can also have an oil dispersed within, or dispersed in, glycerol, liquid polyethylene glycols, and mixtures thereof. Under ordinary conditions of storage and use, these preparations of compound 1 may contain a preservative to prevent the growth of microorganisms. Examples of suitable preservatives include, but are not limited to, benzalkonium chloride, methyl and ethyl parabens, hexetidine, phenyl mercuric salts and the like, and mixtures thereof.

It is believed that pharmaceutical compositions comprising compound 1 may be useful, alone or in combination with other drugs, in the treatment and/or prevention of a wide variety of diseases and disorders, including neurological disorders. Examples of neurological disorders that may be treated, or that may be treated with increased efficacy, by pharmaceutical compositions of compound 1 include, but are not limited to: affective disorders, psychiatric disorders, cerebral function disorders, movement disorders, dementias, motor neuron diseases, neurodegenerative diseases, seizure disorders, and headaches. Affective disorders that may be treated by pharmaceutical compositions of compound 1 include, but are not limited to, treatment resistant bipolar depression, bipolar disorders including cyclothymia, seasonal affective disorder, mood disorders, premenstrual dysphoric disorder (PMDD), panic disorders, attention deficit disorder (ADD), attention deficit disorder with hyperactivity (ADDH), attention deficit/hyperactivity disorder (AD/HD), bipolar and manic conditions, obsessive-compulsive disorder, an eating disorder, anorexia nervosa, bulimia, narcolepsy, chronic fatigue syndrome, premenstrual syndrome, psycho-sexual dysfunction, pseudobulbar affect, and emotional lability. Other disorders that may be treated, or that may be treated with increased efficacy, by pharmaceutical compositions of compound 1 include, but are not limited to: hypertension, scleroderma, Parkinson's disease, dyskinesia associated with Parkinson's disease, cognitive impairment, diabetes, migraine, memory disorders.

1. A method of treating a disease or disorder, comprising administering compound 1 to a human being in need thereof, wherein compound 1 is represented by the formula: 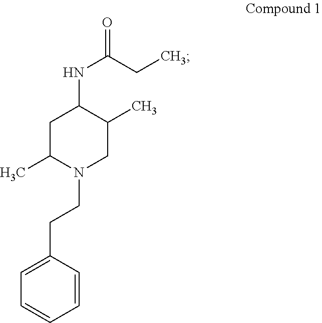 [SMILES: CCC(=O)NC1CC(C)N(CCc2ccccc2)CC1C]
or a pharmaceutically acceptable salt thereof, wherein the disease or disorder is not opioid intoxication, opioid use disorder, opioid overdose, narcotic depression, septic shock, alcoholism, schizophrenia, pain, depression, obesity or weight-gain.

2. The method of claim 1, wherein the disease or disorder is bipolar disorder.

3. The method of claim 1, wherein the disease or disorder is Parkinson's disease.

4. The method of claim 1, wherein the disease or disorder is dyskinesia.

5. The method of claim 1, wherein the disease or disorder is hypertension.

6. The method of claim 1, wherein the disease or disorder is scleroderma.

7. The method of claim 1, wherein the disease or disorder is ADHD.

8. The method of claim 1, wherein compound 1 is administered to the human being once, twice, three times, or four times a day.

9. The method of claim 1, wherein the disease or disorder is a neurological disorder, a psychiatric disorder, or a combination thereof.
